(19) United States
(12) Patent Application Publication
(10) Pub. No.: US 2010/0240131 A1
(43) Pub. Date: Sep. 23, 2010

(54) Method of modifying the genome of gram-positive bacteria by means of a novel conditionally negative dominant marker gene

(75) Inventors: Markus Pompejus, Freinsheim (DE); Burkhard Kroger, Limburgerhof (DE); Hartwig Schroder, Nussloch (DE); Oskar Zelder, Speyer (DE)

Correspondence Address: LAHIVE & COCKFIELD, LLP;FLOOR 30, SUITE 3000, ONE POST OFFICE SQUARE, BOSTON, MA, 02109, US

(73) Assignee: BASF Aktiengesellschaft, Ludwigshafen (DE)

(21) Appl. No.: 11/476,404

(22) Filed: Jun. 28, 2006

Related U.S. Application Data
(63) Continuation of application No. 10/467,479, filed on Aug. 6, 2003, now abandoned.

(30) Foreign Application Priority Data
Mar. 1, 2001 (DE) 101 09 996.7

(51) Int. Cl. C12N 1/21; C12N 15/63; C12N 15/74; C12N 13/00

(52) U.S. Cl. 435/446; 435/320.1; 435/471; 435/252.3

(57) ABSTRACT

The invention relates to a novel method for modifying the genome of Gram-positive bacteria, to these bacteria and to novel vectors. The invention particularly relates to a method for modifying corynebacteria or brevibacteria with the aid of a novel marker gene which has a conditionally negatively dominant action in the bacteria.

The invention relates to a novel method for modifying the genome of Gram-positive bacteria, to these bacteria and to novel vectors. The invention particularly relates to a method for modifying corynebacteria or brevibacteria with the aid of a novel marker gene which has a conditionally negatively dominant action in the bacteria.

Corynebacterium glutamicum is a Gram-positive, aerobic bacterium which (like other corynebacteria, i.e. Corynebacterium and Brevibacterium species too) is used industrially for producing a number of fine chemicals, and also for breaking down hydrocarbons and oxidizing terpenoids (for a review, see, for example, Liebl (1992) “The Genus Corynebacterium”, in: The Procaryotes, Volume II, Balows, A. et al., eds. Springer).

Because of the availability of cloning vectors for use in corynebacteria and techniques for genetic manipulation of C. glutamicum and related Corynebacterium and Brevibacterium species (see, for example, Yoshihama et al., J. Bacteriol. 162 (1985) 591-597; Katsumata et al., J. Bacteriol. 159 (1984) 306-311; and Santamaria et al. J. Gen. Microbiol. 130 (1984) 2237-2246), genetic modification of these organisms is possible (for example by overexpression of genes) in order, for example, to make them better and more efficient as producers of one or more fine chemicals.

The use of plasmids able to replicate in corynebacteria is in this connection a well-established technique which is known to the skilled worker, is widely used and has been documented many times in the literature (see, for example, Deb, J. K et al. (1999) FEMS Microbiol. Lett. 175, 11-20).

It is likewise possible for genetic modification of corynebacteria to take place by modification of the DNA sequence of the genome. It is possible to introduce DNA sequences into the genome (newly introduced and/or introduction of further copies of sequences which are present), it is also possible to delete DNA sequence sections from the genome (e.g. genes or parts of genes), but it is also possible to carry out sequence exchanges (e.g. base exchanges) in the genome.

The modification of the genome can be achieved by introducing into the cell DNA which is preferably not replicated in the cell, and by recombining this introduced DNA with genomic host DNA and thus modifying the genomic DNA. This procedure is described, for example, in van der Rest, M. E. et al. (1999) Appl. Microbiol. Biotechnol. 52, 541-545 and references therein.

It is advantageous to be able to delete the transformation marker used (such as, for example, an antibiotic resistance gene) again because this marker can then be reused in further transformation experiments. One possibility for carrying this out is to use a marker gene which has a conditionally negatively dominant action.

A marker gene which has a conditionally negatively dominant action means, a gene which is disadvantageous (e.g. toxic) for the host under certain conditions but has no adverse effects on the host harboring the gene under other conditions. An example from the literature is the URA3 gene from yeasts or fungi, an essential gene of pyrimidine biosynthesis which, however, is disadvantageous for the host if the chemical 5-fluoroorotic acid is present in the medium (see, for example, DE19801120, Rothstein, R. (1991) Methods in Enzymology 194, 281-301).

The use of a marker gene which has a conditionally negatively dominant action for deleting DNA sequences (for example the transformation marker used and/or vector sequences and other sequence sections), also called “pop-out”, is described, for example, in Schäfer et al. (1994) Gene 145, 69-73 or in Rothstein, R. (1991) Methods in Enzymology 194, 281-301.

The sacB gene from Bacillus subtilis codes for the enzyme levan sucrase (EC 2.4.1.10) and has been described in Steinmetz, M. et al. (1983) Mol. Gen. Genet. 191, 138-144, and Steinmetz, M. et al. (1985) Mol. Gen. Genet. 200, 220-228. It is known (Gay, P. et al. (1985) J. Bacteriology 164, 918-921, Schäfer et al. (1994) Gene 145, 69-73, EP0812918, EP0563527, EP0117823), that the sacB gene from Bacillus subtilis is suitable as a marker gene which has a conditionally negatively dominant action. This selection method is based on the fact that cells which harbor the sacB gene cannot grow in the presence of 5% sucrose. Growth of cells occurs only after loss or inactivation of the levan sucrase. The sensitivity to 10% sucrose of certain Gram-positive bacteria able to express the sacB gene from Bacillus subtilis was then described by Jäger, W. et al. (1992) J. Bacteriology 174, 5462-5465. It has additionally been shown that it is possible with the sacB gene from B. subtilis to carry out in Corynebacterium glutamicum a selection for gene disruptions or an allelic exchange by homologous recombination (Schäfer et al. (1994) Gene 145, 69-73).

It has now been found that the sacB gene from Bacillus amyloliquefaciens (Tang et al. (1990) Gene 96, 89-93) is surprisingly particularly suitable for use as a marker gene which has a conditionally negatively dominant action in corynebacteria. Selectability using sacB depends on the efficiency of expression of the gene in the heterologous host organism. The high efficiency of expression of the sacB gene from B. amyloliquefaciens makes this gene a preferably used gene.

The invention discloses a novel and simple method for modifying genomic sequences in corynebacteria using the sacB gene from Bacillus amyloliquefaciens as novel marker gene which has a conditionally negatively dominant action. This may comprise genomic integrations of nucleic acid molecules (for example complete genes), disruptions (for example deletions or integrative disruptions) and sequence modifications (for example single or multiple point mutations, complete gene exchanges). Preferred disruptions are those leading to a reduction in byproducts of the desired fermentation product, and preferred integrations are those strengthening a desired metabolism into a fermentation product and/or diminishing or eliminating bottlenecks (de-bottlenecking). In the case of sequence modifications, appropriate metabolic adaptations are preferred. The fermentation product is preferably a fine chemical.

The invention relates in particular to a plasmid vector which does not replicate in the target organism, having the following components:
a) an origin of replication for E. coli,
b) one or more genetic markers,
c) optionally a sequence section which enables DNA transfer in particular by conjugation (mob),
d) a sequence section which is homologous to sequences of the target organism and mediates homologous recombination in the target organism,
e) the sacB gene from B. amyloliquefaciens under the control of a promoter.

Target organism means in this connection the organism whose genomic sequence is to be modified.

The invention additionally relates to a method for marker-free mutagenesis in Gram-positive bacterial strains comprising the following steps:
a) provision of a vector as indicated above,
b) transfer of the vector into a Gram-positive bacterium
c) selection for one or more genetic markers
d) selection of one or more clones of transfected Gram-positive bacteria by cultivating the transfected clones in a sucrose-containing medium,
   and a bacterium available by this method as far as step c).

The promoter is preferably heterologous to B. amyloliquefaciens and is, in particular, from E. coli or C. glutamicum and additionally in particular the tac promoter.

Sequences exchanged in the target organism are, in particular, those which increase the yields in the production of fine chemicals. Examples of such genes are indicated in WO 01/0842, 843 & 844, WO 01/0804 & 805, WO 01/2583.

The transfer of DNA into the target organism is made possible in particular by conjugation or electroporation. DNA which is to be transferred by conjugation into the target organism comprises special sequence sections which make this possible. Such so-called mob sequences and their use are described, for example, in Schäfer, A. et al. (1991) J. Bacteriol. 172, 1663-1666.

Genetic marker means a selectable property. Preference is given to antibiotic resistances, in particular a resistance to kanamycin, chloramphenicol, tetracycline or ampicillin.

Sucrose-containing medium means, in particular, a medium with not less than 5% and not more than 10% (by weight) sucrose.

Target organism means the organism which is to be genetically modified by the method of the invention. Preferred meanings are Gram-positive bacteria, in particular bacterial strains from the genus Brevibacterium or Corynebacterium. Corynebacteria means for the purposes of the invention Corynebacterium species, Brevibacterium species and Mycobacterium species. Preference is given to Corynebacterium species and Brevibacterium species. Examples of Corynebacterium species and Brevibacterium species are: Brevibacterium brevis, Brevibacterium lactofermentum, Corynebacterium ammoniagenes, Corynebacterium glutamicum, Corynebacterium diphtheriae, Corynebacterium lactofermentum. Examples of Mycobacterium species are: Mycobacterium tuberculosis, Mycobacterium leprae, Mycobacterium bovis, Mycobacterium smegmatis.

Particular preference is given to the strains indicated in the table below:

TABLE
Corynebacterium and Brevibacterium strains:

| Genus | Species | ATCC | FERM | NRRL | CECT | NCIMB | CBS |
|---|---|---|---|---|---|---|---|
| Brevibacterium | ammoniagenes | 21054 | | | | | |
| Brevibacterium | ammoniagenes | 19350 | | | | | |
| Brevibacterium | ammoniagenes | 19351 | | | | | |
| Brevibacterium | ammoniagenes | 19352 | | | | | |
| Brevibacterium | ammoniagenes | 19353 | | | | | |
| Brevibacterium | ammoniagenes | 19354 | | | | | |
| Brevibacterium | ammoniagenes | 19355 | | | | | |
| Brevibacterium | ammoniagenes | 19356 | | | | | |
| Brevibacterium | ammoniagenes | 21055 | | | | | |
| Brevibacterium | ammoniagenes | 21077 | | | | | |
| Brevibacterium | ammoniagenes | 21553 | | | | | |
| Brevibacterium | ammoniagenes | 21580 | | | | | |
| Brevibacterium | ammoniagenes | 39101 | | | | | |
| Brevibacterium | butanicum | 21196 | | | | | |
| Brevibacterium | divaricatum | 21792 | P928 | | | | |
| Brevibacterium | flavum | 21474 | | | | | |
| Brevibacterium | flavum | 21129 | | | | | |
| Brevibacterium | flavum | 21518 | | | | | |
| Brevibacterium | flavum | | | B11474 | | | |
| Brevibacterium | flavum | | | B11472 | | | |
| Brevibacterium | flavum | 21127 | | | | | |
| Brevibacterium | flavum | 21128 | | | | | |
| Brevibacterium | flavum | 21427 | | | | | |
| Brevibacterium | flavum | 21475 | | | | | |
| Brevibacterium | flavum | 21517 | | | | | |
| Brevibacterium | flavum | 21528 | | | | | |
| Brevibacterium | flavum | 21529 | | | | | |
| Brevibacterium | flavum | | | B11477 | | | |
| Brevibacterium | flavum | | | B11478 | | | |
| Brevibacterium | flavum | 21127 | | | | | |
| Brevibacterium | flavum | | | B11474 | | | |
| Brevibacterium | healii | 15527 | | | | | |
| Brevibacterium | ketoglutamicum | 21004 | | | | | |
| Brevibacterium | ketoglutamicum | 21089 | | | | | |
| Brevibacterium | ketosoreductum | 21914 | | | | | |
| Brevibacterium | lactofermentum | | | | 70 | | |
| Brevibacterium | lactofermentum | | | | 74 | | |
| Brevibacterium | lactofermentum | | | | 77 | | |
| Brevibacterium | lactofermentum | 21798 | | | | | |
| Brevibacterium | lactofermentum | 21799 | | | | | |
| Brevibacterium | lactofermentum | 21800 | | | | | |
| Brevibacterium | lactofermentum | 21801 | | | | | |
| Brevibacterium | lactofermentum | | | B11470 | | | |
| Brevibacterium | lactofermentum | | | B11471 | | | |
| Brevibacterium | lactofermentum | 21086 | | | | | |
| Brevibacterium | lactofermentum | 21420 | | | | | |
| Brevibacterium | lactofermentum | 21086 | | | | | |
| Brevibacterium | lactofermentum | 31269 | | | | | |
| Brevibacterium | linens | 9174 | | | | | |
| Brevibacterium | linens | 19391 | | | | | |
| Brevibacterium | linens | 8377 | | | | | |
| Brevibacterium | paraffinolyticum | | | | | 11160 | |
| Brevibacterium | spec. | | | | | | 717.73 |
| Brevibacterium | spec. | | | | | | 717.73 |
| Brevibacterium | spec. | 14604 | | | | | |
| Brevibacterium | spec. | 21860 | | | | | |
| Brevibacterium | spec. | 21864 | | | | | |
| Brevibacterium | spec. | 21865 | | | | | |
| Brevibacterium | spec. | 21866 | | | | | |
| Brevibacterium | spec. | 19240 | | | | | |
| Corynebacterium | acetoacidophilum | 21476 | | | | | |
| Corynebacterium | acetoacidophilum | 13870 | | | | | |
| Corynebacterium | acetoglutamicum | | | B11473 | | | |
| Corynebacterium | acetoglutamicum | | | B11475 | | | |
| Corynebacterium | acetoglutamicum | 15806 | | | | | |
| Corynebacterium | acetoglutamicum | 21491 | | | | | |
| Corynebacterium | acetoglutamicum | 31270 | | | | | |
| Corynebacterium | acetophilum | | | B3671 | | | |
| Corynebacterium | ammoniagenes | 6872 | | | | | |
| Corynebacterium | ammoniagenes | 15511 | | | | | |
| Corynebacterium | fujiokense | 21496 | | | | | |
| Corynebacterium | glutamicum | 14067 | | | | | |
| Corynebacterium | glutamicum | 39137 | | | | | |
| Corynebacterium | glutamicum | 21254 | | | | | |
| Corynebacterium | glutamicum | 21255 | | | | | |
| Corynebacterium | glutamicum | 31830 | | | | | |
| Corynebacterium | glutamicum | 13032 | | | | | |
| Corynebacterium | glutamicum | 14305 | | | | | |
| Corynebacterium | glutamicum | 15455 | | | | | |
| Corynebacterium | glutamicum | 13058 | | | | | |
| Corynebacterium | glutamicum | 13059 | | | | | |
| Corynebacterium | glutamicum | 13060 | | | | | |
| Corynebacterium | glutamicum | 21492 | | | | | |
| Corynebacterium | glutamicum | 21513 | | | | | |
| Corynebacterium | glutamicum | 21526 | | | | | |
| Corynebacterium | glutamicum | 21543 | | | | | |
| Corynebacterium | glutamicum | 13287 | | | | | |
| Corynebacterium | glutamicum | 21851 | | | | | |
| Corynebacterium | glutamicum | 21253 | | | | | |
| Corynebacterium | glutamicum | 21514 | | | | | |
| Corynebacterium | glutamicum | 21516 | | | | | |
| Corynebacterium | glutamicum | 21299 | | | | | |
| Corynebacterium | glutamicum | 21300 | | | | | |
| Corynebacterium | glutamicum | 39684 | | | | | |
| Corynebacterium | glutamicum | 21488 | | | | | |
| Corynebacterium | glutamicum | 21649 | | | | | |
| Corynebacterium | glutamicum | 21650 | | | | | |
| Corynebacterium | glutamicum | 19223 | | | | | |
| Corynebacterium | glutamicum | 13869 | | | | | |
| Corynebacterium | glutamicum | 21157 | | | | | |
| Corynebacterium | glutamicum | 21158 | | | | | |
| Corynebacterium | glutamicum | 21159 | | | | | |
| Corynebacterium | glutamicum | 21355 | | | | | |
| Corynebacterium | glutamicum | 31808 | | | | | |
| Corynebacterium | glutamicum | 21674 | | | | | |
| Corynebacterium | glutamicum | 21562 | | | | | |
| Corynebacterium | glutamicum | 21563 | | | | | |
| Corynebacterium | glutamicum | 21564 | | | | | |
| Corynebacterium | glutamicum | 21565 | | | | | |
| Corynebacterium | glutamicum | 21566 | | | | | |
| Corynebacterium | glutamicum | 21567 | | | | | |
| Corynebacterium | glutamicum | 21568 | | | | | |
| Corynebacterium | glutamicum | 21569 | | | | | |
| Corynebacterium | glutamicum | 21570 | | | | | |
| Corynebacterium | glutamicum | 21571 | | | | | |
| Corynebacterium | glutamicum | 21572 | | | | | |
| Corynebacterium | glutamicum | 21573 | | | | | |
| Corynebacterium | glutamicum | 21579 | | | | | |
| Corynebacterium | glutamicum | 19049 | | | | | |
| Corynebacterium | glutamicum | 19050 | | | | | |
| Corynebacterium | glutamicum | 19051 | | | | | |
| Corynebacterium | glutamicum | 19052 | | | | | |
| Corynebacterium | glutamicum | 19053 | | | | | |
| Corynebacterium | glutamicum | 19054 | | | | | |
| Corynebacterium | glutamicum | 19055 | | | | | |
| Corynebacterium | glutamicum | 19056 | | | | | |
| Corynebacterium | glutamicum | 19057 | | | | | |
| Corynebacterium | glutamicum | 19058 | | | | | |
| Corynebacterium | glutamicum | 19059 | | | | | |
| Corynebacterium | glutamicum | 19060 | | | | | |
| Corynebacterium | glutamicum | 19185 | | | | | |
| Corynebacterium | glutamicum | 13286 | | | | | |
| Corynebacterium | glutamicum | 21515 | | | | | |
| Corynebacterium | glutamicum | 21527 | | | | | |
| Corynebacterium | glutamicum | 21544 | | | | | |
| Corynebacterium | glutamicum | 21492 | | | | | |
| Corynebacterium | glutamicum | | | B8183 | | | |
| Corynebacterium | glutamicum | | | B8182 | | | |
| Corynebacterium | glutamicum | | | B12416 | | | |
| Corynebacterium | glutamicum | | | B12417 | | | |
| Corynebacterium | glutamicum | | | B12418 | | | |
| Corynebacterium | glutamicum | | | B11476 | | | |
| Corynebacterium | glutamicum | 21608 | | | | | |
| Corynebacterium | lilium | | P973 | | | | |
| Corynebacterium | nitrilophilus | 21419 | | | | 11594 | |
| Corynebacterium | spec. | | P4445 | | | | |
| Corynebacterium | spec. | | P4446 | | | | |
| Corynebacterium | spec. | 31088 | | | | | |
| Corynebacterium | spec. | 31089 | | | | | |
| Corynebacterium | spec. | 31090 | | | | | |
| Corynebacterium | spec. | 31090 | | | | | |
| Corynebacterium | spec. | 31090 | | | | | |
| Corynebacterium | spec. | 15954 | | | | | |
| Corynebacterium | spec. | 21857 | | | | | |
| Corynebacterium | spec. | 21862 | | | | | |
| Corynebacterium | spec. | 21863 | | | | | |

ATCC: American Type Culture Collection, Rockville, MD, USA
FERM: Fermentation Research Institute, Chiba, Japan
NRRL: ARS Culture Collection, Northern Regional Research Laboratory, Peoria, IL, USA
CECT: Coleccion Espanola de Cultivos Tipo, Valencia, Spain
NCIMB: National Collection of Industrial and Marine Bacteria Ltd., Aberdeen, UK
CBS: Centraalbureau voor Schimmelcultures, Baarn, NL

The mutants generated in this way can then be used to produce fine chemicals or, in the case of C. diphtheriae, to produce, for example, vaccines with attenuated or nonpathogenic organisms. Fine chemicals mean: organic acids, both proteinogenic and nonproteinogenic amino acids, nucleotides and nucleosides, lipids and fatty acids, diols, carbohydrates, aromatic compounds, vitamins and cofactors, and enzymes.

The term “fine chemical” is known in the art and comprises molecules which are produced by an organism and are used in various branches of industry such as, for example, but not restricted to, the pharmaceutical industry, the agricultural industry and the cosmetics industry. These compounds comprise organic acids such as tartaric acid, itaconic acid and diaminopimelic acid, both proteinogenic and nonproteinogenic amino acids, purine and pyrimidine bases, nucleosides and nucleotides (as described, for example, in Kuninaka, A. (1996) Nucleotides and related compounds, pp. 561-612, in Biotechnology Vol. 6, Rehm et al., editors VCH: Weinheim and the references therein), lipids, saturated and unsaturated fatty acids (for example arachidonic acid), diols (for example propanediol and butanediol), carbohydrates (for example hyaluronic acid and trehalose), aromatic compounds (for example aromatic amines, vanillin and indigo), vitamins and cofactors (as described in Ullmann's Encyclopedia of Industrial Chemistry, Vol. A27, “Vitamins”, pp. 443-613 (1996) VCH: Weinheim and the references therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held Sep. 1-3, 1994, in Penang, Malaysia, AOCS Press (1995)), Enzymes, Polyketides (Cane et al. (1998) Science 282: 63-68), and all other chemicals described by Gutcho (1983) in Chemicals by Fermentation, Noyes Data Corporation, ISBN: 0818805086 and the references indicated therein. The metabolism and the uses of certain fine chemicals are explained further below.

### A. Amino Acid Metabolism and Uses

Amino acids comprise the fundamental structural units of all proteins and are thus essential for normal functions of the cell. The term “amino acid” is known in the art. Proteinogenic amino acids, of which there are 20 types, serve as structural units for proteins, in which they are linked together by peptide bonds, whereas the nonproteinogenic amino acids (hundreds of which are known) usually do not occur in proteins (see Ullmann's Encyclopedia of Industrial Chemistry, Vol. A2, pp. 57-97 VCH: Weinheim (1985)). Amino acids can exist in the D or L configuration, although L-amino acids are usually the only type found in naturally occurring proteins. Biosynthetic and degradation pathways of each of the 20 proteinogenic amino acids are well characterized both in prokaryotic and eukaryotic cells (see, for example, Stryer, L. Biochemistry, 3^{rd} edition, pp. 578-590 (1988)). The “essential” amino acids (histidine, isoleucine, leucine, lysine, methionine, phenylalanine, threonine, tryptophan and valine), so called because, owing to the complexity of their biosyntheses, they must be taken in with the diet, are converted by simple biosynthetic pathways into the other 11 “nonessential” amino acids (alanine, arginine, asparagine, aspartate, cysteine, glutamate, glutamine, glycine, proline, serine and tyrosine). Higher animals are able to synthesize some of these amino acids but the essential amino acids must be taken in with the food in order that normal protein synthesis takes place.
Apart from their function in protein biosynthesis, these amino acids are interesting chemicals as such, and it has been found that many have various applications in the human food, animal feed, chemicals, cosmetics, agricultural and pharmaceutical industries. Lysine is an important amino acid not only for human nutrition but also for monogastric livestock such as poultry and pigs. Glutamate is most frequently used as flavor additive (monosodium glutamate, MSG) and elsewhere in the food industry, as are aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D/L-methionine are widely used animal feed additives (Leuchtenberger, W. (1996) Amino acids—technical production and use, pp. 466-502 in Rehm et al., (editors) Biotechnology Vol. 6, Chapter 14a, VCH: Weinheim). It has been found that these amino acids are additionally suitable as precursors for synthesizing synthetic amino acids and proteins, such as N-acetylcysteine, S-carboxymethyl-L-cysteine, (S)-5-hydroxytryptophan and other substances described in Ullmann's Encyclopedia of Industrial Chemistry, Vol. A2, pp. 57-97, VCH, Weinheim, 1985.
The biosynthesis of these natural amino acids in organisms able to produce them, for example bacteria, has been well characterized (for a review of bacterial amino acid biosynthesis and its regulation, see Umbarger, H. E. (1978) Ann. Rev. Biochem. 47: 533-606). Glutamate is synthesized by reductive amination of α-ketoglutarate, an intermediate product in the citric acid cycle. Glutamine, proline and arginine are each generated successively from glutamate. The biosynthesis of serine takes place in a three-step process and starts with β-phosphoglycerate (an intermediate product of glycolysis), and affords this amino acid after oxidation, transamination and hydrolysis steps. Cysteine and glycine are each produced from serine, specifically the former by condensation of homocysteine with serine, and the latter by transfer of the side-chain β-carbon atom to tetrahydrofolate in a reaction catalyzed by serine transhydroxymethylase. Phenylalanine and tyrosine are synthesized from the precursors of the glycolysis and pentose phosphate pathway, and erythrose 4-phosphate and phosphoenolpyruvate in a 9-step biosynthetic pathway which diverges only in the last two steps after the synthesis of prephenate. Tryptophan is likewise produced from these two starting molecules but it is synthesized by an 11-step pathway. Tyrosine can also be prepared from phenylalanine in a reaction catalyzed by phenylalanine hydroxylase. Alanine, valine and leucine are each biosynthetic products derived from pyruvate, the final product of glycolysis. Aspartate is formed from oxalacetate, an intermediate product of the citrate cycle. Asparagine, methionine, threonine and lysine are each produced by the conversion of aspartate. Isoleucine is formed from threonine. Histidine is formed from 5-phosphoribosyl 1-pyrophosphate, an activated sugar, in a complex 9-step pathway.
Amounts of amino acids exceeding those required for protein biosynthesis by the cell cannot be stored and are instead broken down so that intermediate products are provided for the principal metabolic pathways in the cell (for a review, see Stryer, L., Biochemistry, 3^{rd} edition, Chapter 21 “Amino Acid Degradation and the Urea Cycle”; pp. 495-516 (1988)). Although the cell is able to convert unwanted amino acids into the useful intermediate products of metabolism, production of amino acids is costly in terms of energy, the precursor molecules and the enzymes necessary for their synthesis. It is therefore not surprising that amino acid biosynthesis is regulated by feedback inhibition, whereby the use of a particular amino acid slows down or completely stops its own production (for a review of the feedback mechanism in amino acid biosynthetic pathways, see Stryer, L., Biochemistry, 3^{rd} edition, Chapter 24, “Biosynthesis of Amino Acids and Heme”, pp. 575-600 (1988)). The output of a particular amino acid is therefore restricted by the amount of this amino acid in the cell.

### B. Vitamins, Cofactors and Nutraceutical Metabolism, and Uses

Vitamins, cofactors and nutraceuticals comprise another group of molecules. Higher animals have lost the ability to synthesize them and therefore have to take them in, although they are easily synthesized by other organisms such as bacteria. These molecules are either bioactive molecules per se or precursors of bioactive substances which serve as electron carriers or intermediate products in a number of metabolic pathways. Besides their nutritional value, these compounds also have a significant industrial value as colorants, antioxidants and catalysts or other processing auxiliaries. (For a review of the structure, activity and industrial applications of these compounds, see, for example, Ullmann's Encyclopedia of Industrial Chemistry, “Vitamins”, Vol. A27, pp. 443-613, VCH: Weinheim, 1996). The term “vitamin” is known in the art and comprises nutrients which are required for normal functional of an organism but cannot be synthesized by this organism itself. The group of vitamins may include cofactors and nutraceutical compounds. The term “cofactor” comprises nonproteinaceous compounds necessary for the appearance of a normal enzymic activity. These compounds may be organic or inorganic; the cofactor molecules of the invention are preferably organic. The term “nutraceutical” comprises food additives which are health-promoting in plants and animals, especially humans. Examples of such molecules are vitamins, antioxidants and likewise certain lipids (e.g. polyunsaturated fatty acids).
The biosynthesis of these molecules in organisms able to produce them, such as bacteria, has been comprehensively characterized (Ullmann's Encyclopedia of Industrial Chemistry, “Vitamins”, Vol. A27, pp. 443-613, VCH: Weinheim, 1996, Michal, G. (1999) Biochemical Pathways: An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research—Asia, held on Sep. 1-3, 1994, in Penang, Malaysia, AOCS Press, Champaign, Ill. X, 374 S).
Thiamine (vitamin B₁) is formed by chemical coupling of pyrimidine and thiazole units. Riboflavin (vitamin B₂) is synthesized from guanosine 5′-triphosphate (GTP) and ribose 5′-phosphate. Riboflavin in turn is employed for the synthesis of flavin mononucleotide (FMN) and flavin adenine dinucleotide (FAD). The family of compounds together referred to as “vitamin B6” (for example pyridoxine, pyridoxamine, pyridoxal 5′-phosphate and the commercially used pyridoxine hydrochloride), are all derivatives of the common structural unit 5-hydroxy-6-methylpyridine. Panthothenate (pantothenic acid, R-(+)-N-(2,4-dihydroxy-3,3-dimethyl-1-oxobutyl)-β-alanine) can be prepared either by chemical synthesis or by fermentation. The last steps in pantothenate biosynthesis consist of ATP-driven condensation of β-alanine and pantoic acid. The enzymes responsible for the biosynthetic steps for the conversion into pantoic acid and into β-alanine and for the condensation to pantothenic acid are known. The metabolically active form of pantothenate is coenzyme A whose biosynthesis takes place by 5 enzymatic steps. Pantothenate, pyridoxal 5′-phosphate, cysteine and ATP are the precursors of coenzyme A. These enzymes catalyze not only the formation of pantothenate but also the production of (R)-pantoic acid, (R)-pantolactone, (R)-panthenol (provitamin B₅), pantetheine (and its derivatives) and coenzyme A.
The biosynthesis of biotin from the precursor molecule pimeloyl-CoA in microorganisms has been investigated in detail, and several of the genes involved have been identified. It has emerged that many of the corresponding proteins are involved in the Fe cluster synthesis and belong to the class of nifS proteins. Liponic acid is derived from octanoic acid and serves as coenzyme in energy metabolism where it is a constituent of the pyruvate dehydrogenase complex and of the α-ketoglutarate dehydrogenase complex. Folates are a group of substances all derived from folic acid which in turn is derived from L-glutamic acid, p-aminobenzoic acid and 6-methylpterin. The biosynthesis of folic acid and its derivatives starting from the metabolic intermediate products of the biotransformation of guanosine 5′-triphosphate (GTP), L-glutamic acid and p-aminobenzoic acid has been investigated in detail in certain microorganisms.
Corrinoids (such as the cobalamines and, in particular, vitamin B₁₂) and the porphyrins belong to a group of chemicals distinguished by a tetrapyrrole ring system. The biosynthesis of vitamin B₁₂ is so complex that it has not yet been completely characterized, but many of the enzymes and substrates involved are now known. Nicotinic acid (nicotinate) and nicotinamide are pyridine derivatives which are also referred to as “niacin”. Niacin is the precursor of the important coenzymes NAD (nicotinamide adenine dinucleotide) and NADP (nicotinamide adenine dinucleotide phosphate) and their reduced forms.
Production of these compounds on the industrial scale is mostly based on cell-free chemical syntheses, although some of these chemicals have likewise been produced by large-scale cultivation of microorganisms, such as riboflavin, vitamin B₆, pantothenate and biotin. Only vitamin B₁₂ is, because of the complexity of its synthesis, produced only by fermentation. In vitro processes require a considerable expenditure of materials and time and frequently high costs.

### C. Purine, Pyrimidine, Nucleoside and Nucleotide Metabolism and Uses

Genes for purine and pyrimidine metabolism and their corresponding proteins are important aims for the therapy of oncoses and viral infections. The term “purine” or “pyrimidine” comprises nitrogen-containing bases which form part of nucleic acids, coenzymes and nucleotides. The term “nucleotide” encompasses the fundamental structural units of nucleic acid molecules, which comprise a nitrogen-containing base, a pentose sugar (the sugar is ribose in the case of RNA and the sugar is D-deoxyribose in the case of DNA) and phosphoric acid. The term “nucleoside” comprises molecules which serve as precursors of nucleotides but have, in contrast to the nucleotides, no phosphoric acid unit. It is possible to inhibit RNA and DNA synthesis by inhibiting the biosynthesis of these molecules or their mobilization to form nucleic acid molecules; targeted inhibition of this activity in cancerous cells allows the ability of tumor cells to divide and replicate to be inhibited.
There are also nucleotides which do not form nucleic acid molecules but serve as energy stores (i.e. AMP) or as coenzymes (i.e. FAD and NAD).
Several publications have described the use of these chemicals for these medical indications, the purine and/or pyrimidine metabolism being influenced (for example Christopherson, R. I. and Lyons, S. D. (1990) “Potent inhibitors of de novo pyrimidine and purine biosynthesis as chemotherapeutic agents”, Med. Res. Reviews 10: 505-548). Investigations of enzymes involved in purine and pyrimidine metabolism have concentrated on the development of novel medicaments which can be used, for example, as immunosuppressants or antiproliferative agents (Smith, J. L. “Enzymes in Nucleotide Synthesis” Curr. Opin. Struct. Biol. 5 (1995) 752-757; Simonds, H. A., Biochem. Soc. Transact. 23 (1995) 877-902). However, purine and pyrimidine bases, nucleosides and nucleotides also have other possible uses: as intermediate products in the biosynthesis of various fine chemicals (e.g. thiamine, S-adenosylmethionine, folates or riboflavin), as energy carriers for the cell (for example ATP or GTP) and for chemicals themselves, are ordinarily used as flavor enhancers (for example IMP or GMP) or for many medical applications (see, for example, Kuninaka, A., (1996) “Nucleotides and Related Compounds in Biotechnology” Vol. 6, Rehm et al., editors VCH: Weinheim, pp. 561-612). Enzymes involved in purine, pyrimidine, nucleoside or nucleotide metabolism are also increasingly serving as targets against which chemicals are being developed for crop protection, including fungicides, herbicides and insecticides.
The metabolism of these compounds in bacteria has been characterized (for reviews, see, for example, Zalkin, H. and Dixon, J. E. (1992) “De novo purine nucleotide biosynthesis” in Progress in Nucleic Acids Research and Molecular biology, Vol. 42, Academic Press, pp. 259-287; and Michal, G. (1999) “Nucleotides and Nucleosides”; Chapter 8 in: Biochemical Pathways: An Atlas of Biochemistry and Molecular Biology, Wiley, New York). Purine metabolism, the object of intensive research, is essential for normal functioning of the cell. Disordered purine metabolism in higher animals may cause severe illnesses, for example gout. Purine nucleotides are synthesized from ribose 5-phosphate by a number of steps via the intermediate compound inosine 5′-phosphate (IMP), leading to the production of guanosine 5′-monophosphate (GMP) or adenosine 5′-monophosphate (AMP), from which the triphosphate forms used as nucleotides can easily be prepared. These compounds are also used as energy stores, so that breakdown thereof provides energy for many different biochemical processes in the cell. Pyrimidine biosynthesis takes place via formation of uridine 5′-monophosphate (UMP) from ribose 5-phosphate. UMP in turn is converted into cytidine 5′-triphosphate (CTP). The deoxy forms of all nucleotides are prepared in a one-step reduction reaction from the diphosphate ribose form of the nucleotide to give the diphosphate deoxyribose form of the nucleotide. After phosphorylation, these molecules can take part in DNA synthesis.

### D. Trehalose Metabolism and Uses

Trehalose consists of two glucose molecules linked together by α,α-1,1 linkage. It is ordinarily used in the food industry as sweetener, as additive for dried or frozen foods and in beverages. However, it is also used in the pharmaceutical industry or in the cosmetics industry and biotechnology industry (see, for example, Nishimoto et al., (1998) U.S. Pat. No. 5,759,610; Singer, M. A. and Lindquist, S. Trends Biotech. 16 (1998) 460-467; Paiva, C. L. A. and Panek, A. D. Biotech Ann. Rev. 2 (1996) 293-314; and Shiosaka, M. J. Japan 172 (1997) 97-102). Trehalose is produced by enzymes of many microorganisms and is naturally released into the surrounding medium from which it can be isolated by methods known in the art.

This procedure can also be carried out with other bacteria in an analogous manner.

### EXAMPLE 1

### Preparation of the Genomic DNA from Bacillus amyloliquefaciens ATCC 23844

A culture of B. amyloliquefaciens ATCC 23844 was grown in Erlenmeyer flasks with LB medium at 37° C. overnight. The bacteria were then pelleted by centrifugation. 1 g of moist cell pellet was resuspended in 2 ml of water, and 260 μl of this were transferred into blue Hybrid matrix tubes, #RYM-61111 (Genome Star Kit, #GC-150). These tubes already contained: 650 μl of phenol (equilibrated with TE buffer, pH 7.5); 650 μl of buffer 1 from the above kit; 130 μl of chloroform. The cells were disrupted in a Ribolyser (Hybaid, #6000220/110) at rotation setting 4.0 for 15 sec and then centrifuged at 4° C. and 10,000 rpm for 5 min. 650 μL of the supernatant were then transferred into 2.0 ml Eppendorf vessels and mixed with 2 μL of RNAse (10 mg/ml). Incubation was then carried out at 37° C. for 60 min. 1/10 volume of 3M Na acetate pH 5.5 and 2 volumes of 100% ethanol were then added to this solution, and it was cautiously mixed. The DNA was then precipitated by centrifugation at 4° C. and 13,000 rpm for 10 minutes. The pellet was washed with 70% ethanol and dried in air. After drying, the DNA pellet was taken up in water and measured by photometry.

### EXAMPLE 2

### PCR Cloning of the Gene for Levan Sucrase (sacB) from Bacillus amyloliquefaciens ATCC 23844

The primer oligonucleotides which can be used for cloning the gene for levan sucrase from Bacillus amyloliquefaciens (ATCC23844) by PCR are those which can be defined on the basis of published sequences for levan sucrase (for example Genbank entry X52988). The PCR can be carried out by methods well known to the skilled worker and described, for example, in Sambrook, J. et al. (1989) “Molecular Cloning: A Laboratory Manual”, Cold Spring Harbor Laboratory Press or Ausubel, F. M. et al. (1994) “Current Protocols in Molecular Biology”, John Wiley & Sons. The gene for levan sucrase (sacB gene), consisting of the protein-coding sequence and 17 by 5′ (ribosome binding site) of the coding sequence can be provided during the PCR with terminal cleavage sites for restriction endonucleases (for example BamHI) and then the PCR product can be cloned into suitable vectors (such as the E. coli plasmid pUC18) which have suitable cleavage sites for restriction endonucleases. This method of cloning genes by PCR is known to the skilled worker and described, for example, in Sambrook, J. et al. (1989) “Molecular Cloning: A Laboratory Manual”, Cold Spring Harbor Laboratory Press or Ausubel, F. M. et al. (1994) “Current Protocols in Molecular Biology”, John Wiley & Sons. It can be demonstrated by sequence analysis (as described in Example 3) that the sacB gene from B. amyloliquefaciens has been cloned with the known sequence. The following primers were employed for the PCR reaction:

Primer 1:
5′-GCGGCCGCCAGAAGGAGACATGAACATGAACATCAAAAAATTGTAAA
ACAAGCC-3′
Primer 2:
5′-ACTAGTTTAGTTGACTGTCAGCTGTCC-3′

### EXAMPLE 3

### Testing of the sacB-Mediated Sucrose Sensitivity in Corynebacterium glutamicum ATCC13032

The sacB gene from B. amyloliquefaciens was initially put under the control of a heterologous promoter. For this purpose, the tac promoter from E. coli was cloned by PCR methods as described in Example 2. The following primers were used for this:

| | |
|---|---|
| Primer 3: | 5′-GGTACCGTTCTGGCAAATATTCTGAAATGAGC-3′ |
| Primer 4: | 5′-GCGGCCGCTTCTGTTTCCTGTGTGAAATTG-3′ |

The tac promoter and the sacB gene were then fused via the common NotI restriction endonuclease cleavage site and cloned by means of the AspI and SpeI cleavage sites in a shuttle vector which is replicable both in E. coli and in C. glutamicum and confers kanamycin resistance. After DNA transfer to C. glutamicum (see, for example, WO 01/02583) and selection of kanamycin-resistant colonies, about 20 of these colonies were streaked in parallel on agar plates containing either 10% sucrose or no sucrose. CM plates (10 g/l glucose, 2.5 g/l NaCl, 2 g/l urea, 10 g/l polypeptone, 5 g/l yeast extract, 5 g/1 meat extract, 22 g/l agar, pH 6.8 with 2 M NaOH, per plate: 4 μL of IPTG 26% strength) were suitable for this selection and were incubated at 30° C. Clones with expressed sacB gene were grown on overnight only on sucrose-free plates.

### EXAMPLE 4

### Inactivation of the ddh Gene from Corynebacterium glutamicum

Any suitable sequence section at the 5′ end of the ddh gene of C. glutamicum (Ishino et al. (1987) Nucleic Acids Res. 15, 3917) and any suitable sequence section at the 3′ end of the ddh gene can be amplified by known PCR methods. The two PCR products can be fused by known methods so that the resulting product has no functional ddh gene. This inactive form of the ddh gene, and the sacB gene from B. amyloliquefaciens, can be cloned into pSL18 (Kim, Y. H. & H.-S. Lee (1996) J. Microbiol. Biotechnol. 6, 315-320) to result in the vector pSL18sacBaΔddh. The procedure is familiar to the skilled worker. Transfer of this vector into Corynebacterium is known to the skilled worker and is possible, for example, by conjugation or electroporation.

Selection of the integrants can take place with kanamycin, and selection for the “pop-out” can take place as described in Example 2. Inactivation of the ddh gene can be shown, for example, by the lack of Ddh activity. Ddh activity can be measured by known methods (see, for example, Misono et al. (1986) Agric. Biol. Chem. 50, 1329-1330).

We claim:

1. A plasmid vector which does not replicate in the target organism, having the following components:
a) an origin of replication for E. coli,
b) one or more genetic markers,
c) optionally a sequence section which enables DNA transfer by conjugation (mob),
d) a sequence section which is homologous to sequences of the target organism and mediates homologous recombination in the target organism,
e) the sacB gene from B. amyloliquefaciens under the control of a promoter.

2. A plasmid vector as claimed in the preceding claim, where the genetic marker mediates an antibiotic resistance.

3. A plasmid vector as claimed in either of the preceding claims, where the promoter is heterologous.

4. A plasmid vector as claimed in any of the preceding claims, where component c) is present.

5. A plasmid vector as claimed in any of the preceding claims, where the antibiotic resistance is a kanamycin, chloramphenicol, tetracycline or ampicillin resistance.

6. A plasmid vector as claimed in any of the preceding claims, where the heterologous promoter originates from E. coli or C. glutamicum.

7. A plasmid vector as claimed in any of the preceding claims, where the heterologous promoter is the tac promoter.

8. A method for the marker-free mutagenesis in a Gram-positive bacterial strain comprising the following steps:
a) provision of a vector as claimed in claim 1,
b) transfer of the vector into a Gram-positive bacterium
c) selection for one or more genetic markers
d) selection of one or more clones of transfected Gram-positive bacteria by cultivating the transfected clones in a sucrose-containing medium.

9. A method as claimed in the preceding claim, where the Gram-positive bacterial strain originates from the genus Brevibacterium or Corynebacterium.

10. A method as claimed in either of the preceding claims, where the DNA transfer takes place by conjugation or electroporation.

11. A bacterium obtainable by a method of claims 8 to 10 as far as step c).


SEQUENCE LISTING (6 sequences)

SEQ ID NO: 1 - DNA, 2350 bp, Bacillus amyloliquefaciens
gaattccttcaggaaaagaacgatggctgtcttattagcggttgcaggcacatttattttggtcacacacgggaatgtcggcagcctgtctatatccggtctggctgttttttggggcatcagctcggcatttgcgctggcgttttacaccctccagccgcatcggcttttgaagaaatggggctccgccattattgtcggatggggcatgctgatgcggagccgttctcagcctgattcagccgccttggaagtttgaaggccaatggtcgttgtccgcatatgccgcgatcgtgtttatcatcattttcggaacgctcatcgctttttattgctatttggaaagcctgaaatatctgagtgcctctgaaaccagcctcctcgcctgtgcagagccgctgtcagcagcttttttagcggtgatctggctgcatgttcccttcggaatatcagaatggctgggtactttactgattttagccaccatcgctttattatctatcaagaaaaaataacctctcttttttagagaggtttttccctaggcctgaagcaccctttagtctcaattacccataaattaaaaggccttttttcgttttactatcattcaaaagaggaaaatagaccagttgtcaatagaatcagagtctaatagaatgaggtcgaaaagtaaatcacgcaggattgttactgataaagcaggcaagacctaaaatgtgttaagggcaaagtgtattctttggcgtcatcccttacatattttgggtctttttttctgtaacaaacctgccatccatgaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttggcccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattc

SEQ ID NO: 2 - protein, 472 aa, Bacillus amyloliquefaciens
MNIKKIVKQATVLTFTTALLAGGATQAFAKENNQKAYKETYGVSHITRHDMLQIPKQQQNEKYQVPQFDQSTIKNIESAKGLDVWDSWPLQNADGTVAEYNGYHVVFALAGSPKDADDTSIYMFYQKVGDNSIDSWKNAGRVFKDSDKFDANDPILKDQTQEWSGSATFTSDGKIRLFYTDYSGKHYGKQSLTTAQVNVSKSDDTLKINGVEDHKTIFDGDGKTYQNVQQFIDEGNYTSGDNHTLRDPHYVEDKGHKYLVFEANTGTENGYQGEESLFNKAYYGGGTNFFRKESQKLQQSAKKRDAELANGALGIIELNNDYTLKKVMKPLITSNTVTDEIERANVFKMNGKWYLFTDSRGSKMTIDGINSNDIYMLGYVSNSLTGPYKPLNKTGLVLQMGLDPNDVTFTYSHFAVPQAKGNNVVITSYMTNRGFFEDKKATFGPSFLMNIKGNKTSVVKNSILEQGQLTVN

SEQ ID NO: 3 - DNA, 54 bp, Artificial Sequence
  Primer
gcggccgccagaaggagacatgaacatgaacatcaaaaaattgtaaaacaagcc

SEQ ID NO: 4 - DNA, 27 bp, Artificial Sequence
  Primer
actagtttagttgactgtcagctgtcc

SEQ ID NO: 5 - DNA, 32 bp, Artificial Sequence
  Primer
ggtaccgttctggcaaatattctgaaatgagc

SEQ ID NO: 6 - DNA, 30 bp, Artificial Sequence
  Primer
gcggccgcttctgtttcctgtgtgaaattg